(19) United States
(12) Patent Application Publication
(10) Pub. No.: US 2024/0336693 A1
(43) Pub. Date: Oct. 10, 2024

(54) BIOMARKERS FOR CD40 AGONIST THERAPY

(71) Applicant: Apexigen, Inc., Boston, MA (US)

(72) Inventors: Xiaodong Yang, Palo Alto, CA (US); Yuliya Katlinskaya, Wayland, MA (US); Erin L. Filbert, Menlo Park, CA (US)

(73) Assignee: Apexigen, Inc., Boston, MA (US)

(21) Appl. No.: 18/293,997

(22) PCT Filed: Aug. 9, 2022
(86) PCT No.: PCT/US22/74696

§ 371 Date: Jan. 31, 2024

Related U.S. Application Data
(60) Provisional application No. 63/231,428, filed on Aug. 10, 2021.

(51) Int. Cl. C07K 16/28; A61K 39/00; A61P 35/00

(52) U.S. Cl. CPC C07K 16/2878; A61P 35/00; C07K 16/2818; A61K 2039/505; C07K 2317/75

(57) ABSTRACT

Provided are biomarkers and other characteristics for predicting tumor responsiveness to CD40 agonist therapy in certain cancers, including melanoma, PD-L1 negative melanoma, and PD-1 refractory melanoma. Further provided are methods of using these biomarkers and characteristics to identify individuals having cancer for treatment with a CD40 agonist therapy, and subsequently treating these individuals with a CD40 agonist therapy, and related kits and compositions.

### CROSS-REFERENCE TO RELATED APPLICATIONS

This application is a U.S. National Stage application under 35 U.S.C. § 371 of International Application No. PCT/US2022/074696, having a filing date of Aug. 9, 2022, and published in English, which claims the benefit under 35 U.S.C. § 119 (e) of U.S. Provisional Application No. 63/231,428, filed Aug. 10, 2021, all of which are incorporated by reference in their entireties for all purposes.

### BACKGROUND

### Technical Field

The present disclosure relates to biomarkers and other characteristics for predicting tumor responsiveness to CD40 agonist therapy, and related kits, compositions, and methods of treating melanomas, including PD-1 refractory melanomas.

### Description of the Related Art

Activation of CD40 signaling has been shown to directly inhibit tumors, rescue the function of antigen-presenting cells in tumor-bearing hosts, and trigger or restore active immune responses against tumor-associated antigens. CD40 agonist therapy has been reported to overcome T-cell tolerance in tumor-bearing mice, evoke effective cytotoxic T-cell responses against tumor-associated antigens, and enhance the efficacy of anti-tumor vaccines (see, for example, Djureinovic et al., Cancers (Basel). 13:1302, 2021; Eliopoulos et al., Mol Cell Biol. 20:5503-15, 2000; Tong et al., Clin Cancer Res. 7:691-703, 2001).

However, there is a need in the art to better predict tumor responsiveness to CD40 agonist therapy, and thereby select patients that will benefit most from treatment with this therapeutic modality.

### BRIEF SUMMARY

Embodiments of the present disclosure include methods of treating melanoma in a human subject in need thereof, comprising administering to the subject a CD40 agonist, wherein the subject has increased soluble CD8 (sCD8) levels and decreased CCL17 levels, relative to a control or reference; thereby treating melanoma in the human subject in need thereof. In some embodiments, the subject has increased sCD8 serum protein levels and decreased CCL17 1 serum protein levels, relative to a control or reference.

Certain embodiments comprise (a) determining sCD8 and CCL17 levels in a tissue sample from the subject, and (b) administering the CD40 agonist to the subject if sCD8 levels in the tissue sample are increased and CCL17 levels in the tissue sample are decreased, relative to a control or reference. In certain embodiments, (a) comprises determining sCD8 and CCL17 protein levels in the tissue sample by an immunoassay, optionally by a high-throughput multiplex immunoassay, immunohistochemistry (IHC) such as chromogenic or fluorescent IHC, enzyme linked immunosorbent assay (ELISA), or Western blot.

In some embodiments, the tissue sample is a liquid biopsy optionally a blood or serum sample, a surgical sample, or other biopsy sample obtained from the subject, optionally a biopsy of melanoma tissue. Certain embodiments comprise obtaining or receiving the tissue sample from the subject in need thereof.

Specific embodiments comprise (a) determining sCD8 and CCL17 protein levels in a serum sample from the subject by a high-throughput multiplex immunoassay, and (b) administering the CD40 agonist to the subject if sCD8 protein levels in the serum sample are increased and CCL17 protein levels in the serum sample are decreased, relative to a control or reference.

In some embodiments, the CD40 agonist is selected from sotigalimab (APX005M), ChiLob7/4, ADC-1013, SEA-CD40, selicrelumab, and CDX-1140. In some embodiments, the melanoma is PD-1 refractory melanoma.

Also included are methods of treating melanoma in a human subject in need thereof, comprising administering to the subject a CD40 agonist, wherein the subject has increased CD4, CD40, and CCL14 levels and decreased ABCF1, TP53, TCF3, and HDAC11 levels, relative to a control or reference, thereby treating melanoma in the human subject in need thereof. In some embodiments, the subject has increased CD4, CD40, and CCL14 RNA levels and decreased ABCF1, TP53, TCF3, and HDAC11 RNA levels in melanoma tissue, relative to a control or reference.

Certain embodiments comprise (a) determining CD4, CD40, CCL14, ABCF1, TP53, TCF3, and HDAC11 levels in a tissue sample from the subject, and (b) administering the CD40 agonist to the subject if CD4, CD40, and CCL14 levels in the tissue sample are increased and ABCF1, TP53, TCF3, and HDAC11 levels in the tissue sample are decreased, relative to a control or reference. In some embodiments, (a) comprises determining CD4, CD40, CCL14, ABCF1, TP53, TCF3, and HDAC11 RNA expression levels, optionally by a nucleic acid hybridization assay (such as sandwich hybridization assay, competitive hybridization assay, nuclease hybridization assay, hybridization-ligation assay, or dual ligation hybridization assay) or RNA sequencing (RNA-Seq). In some embodiments, (a) comprises determining CD4, CD40, CCL14, ABCF1, TP53, TCF3, and HDAC11 protein levels, optionally by an immunoassay, optionally by a high-throughput multiplex immunoassay, immunohistochemistry (IHC) such chromogenic or fluorescent IHC, enzyme linked immunosorbent assay (ELISA), or Western blot.

In some embodiments, the tissue sample is a liquid biopsy optionally a blood or serum sample, a surgical sample, or other biopsy sample obtained from the subject, optionally a biopsy of melanoma tissue, including a formalin fixed paraffin embedded (FFPE) tissue sample. Certain embodiments comprise obtaining or receiving the tissue sample from the subject in need thereof.

Specific embodiments comprise (a) determining CD4, CD40, CCL14, ABCF1, TP53, TCF3, and HDAC11 RNA expression levels in a melanoma biopsy sample from the subject, optionally a FFPE biopsy sample, and (b) administering the CD40 agonist to the subject if CD4, CD40, and CCL14 levels in the tissue sample are increased and ABCF1, TP53, TCF3, and HDAC11 levels in the tissue sample are decreased, relative to a control or reference.

In some embodiments, the CD40 agonist is selected from sotigalimab (APX005M), ChiLob7/4, ADC-1013, SEA-CD40, selicrelumab, and CDX-1140. In some embodiments, the melanoma is PD-1 refractory melanoma.

Particular embodiments include methods of treating PD-L1 negative melanoma in a human subject in need thereof, comprising administering to the subject a CD40 agonist optionally in combination with a PD-1/PD-L1 inhibitor, thereby treating the PD-L1 negative melanoma in the human subject in need thereof. Certain embodiments comprise (a) determining PD-L1 levels in a tissue sample from the subject, and (b) administering the CD40 agonist and optional PD-1/PD-L1 inhibitor to the subject if PD-L1 levels in the tissue sample are absent (undetectable) or decreased relative to a control or reference. In some embodiments, (a) comprises determining PD-L1 levels in the tissue sample by an immunoassay, optionally immunohistochemistry (IHC) such as chromogenic or fluorescent IHC, enzyme linked immunosorbent assay (ELISA), or Western blot, or by flow cytometry.

In some embodiments, the tissue sample is a liquid biopsy optionally a blood or serum sample, a surgical sample, or other biopsy sample obtained from the subject, optionally a biopsy of melanoma tissue, including a formalin fixed paraffin embedded (FFPE) tissue sample. Particular embodiments comprise obtaining or receiving the tissue sample from the subject in need thereof.

Specific embodiments comprise (a) determining PD-L1 levels by IHC in a melanoma biopsy sample from the subject, and (b) administering the CD40 agonist and optional PD-1/PD-L1 inhibitor to the subject if PD-L1 levels in the melanoma biopsy sample are absent (undetectable) or decreased relative to a control or reference.

In some embodiments, the CD40 agonist is selected from sotigalimab (APX005M), ChiLob7/4, ADC-1013, SEA-CD40, selicrelumab, and CDX-1140. In some embodiments, the PD-1 inhibitor is selected from nivolumab, pembrolizumab, cemiplimab, JTX-4014, spartalizumab, camrelizumab, sintilimab, tislelizumab, toripalimab, dostarlimab, MGA012, AMP-22, and AMP-514. In some embodiments, the PD-L1 inhibitor is selected from atezolizumab, avelumab, and durvalumab. In some embodiments, the melanoma is PD-1 refractory melanoma.

Also included are methods of treating melanoma in a human subject in need thereof, comprising administering to the subject a CD40 agonist, wherein the subject has previously undergone, and is no longer undergoing, treatment with a CTLA-4 inhibitor, thereby treating the melanoma in the human subject in need thereof.

Some embodiments comprise administering the CD40 agonist at least about 1, 2, 3, 4, 5, 10, 20, 30, 40, 50, 60, 70, 80, 90, 80, 90, 100 days or more after the end of treatment with the CTLA-4 inhibitor, for example, between about 100-200 days, 100-300 days, 100-400 days, 100-500 days, or 100-600 days after the end of treatment with the CTLA-4 inhibitor. Some embodiments comprise administering the CD40 agonist at least about 200 days or more after the end of treatment with the CTLA-4 inhibitor, for example, between about 200-300 days, 200-400 days, 200-500 days, or 200-600 days after the end of treatment with the CTLA-4 inhibitor. Certain embodiments comprise administering the CD40 agonist at least about 300 days or more after the end of treatment with the CTLA-4 inhibitor, for example, between about 300-400 days, 300-500 days, or 300-600 days after the end of treatment with the CTLA-4 inhibitor. Particular embodiments comprise administering the CD40 agonist at least about 400 days or more after the end of treatment with the CTLA-4 inhibitor, for example, between about 400-500 days or 400-600 days after the end of treatment with the CTLA-4 inhibitor. Some embodiments comprise administering the CD40 agonist at least about 500 days or more after the end of treatment with the CTLA-4 inhibitor, for example, between about 500-600 days after the end of treatment with the CTLA-4 inhibitor. Some embodiments comprise administering the CD40 agonist at least about 600 days or more after the end of treatment with the CTLA-4 inhibitor. In some embodiments, prior treatment with the CTLA-4 inhibitor increases the response rate to the CD40 agonist, relative to no prior treatment with a CTLA-4 inhibitor.

In certain embodiments, the CD40 agonist is selected from sotigalimab (APX005M), ChiLob7/4, ADC-1013, SEA-CD40, selicrelumab, and CDX-1140. In certain embodiments, the CTLA inhibitor is selected from ipilimumab and tremelimumab. In specific embodiments, the melanoma is PD-1 refractory melanoma.

### BRIEF DESCRIPTION OF THE DRAWINGS

FIGS. 1A-1B show that increased sCD8A (1A) and decreased CCL17 (1B) correlate with tumor responsiveness to sotigalimab therapy (R, responder; NR, non-responder)

FIG. 2 shows the immunoassay format for analyzing serum protein levels.

FIGS. 3A-3O show that increased expression of CD40 (3A), CD4 (3C), and CCL14 (3O) and decreased expression of TCF3 (3D), TP53 (3F), ABCF1 (3J), and HDAC11 (3M) correlate with tumor responsiveness to sotigalimab therapy.

FIG. 4 shows the tumor responsiveness in subjects with PD-1 refractory melanoma having undergone prior therapy with nivolumab or pembrolizumab.

FIG. 5 shows that prior anti-CTLA4 (Ipilimumab) therapy/proximity correlates with tumor responsiveness to sotigalimab therapy.

### DETAILED DESCRIPTION

Embodiments of the present disclosure relate generally to methods of treating melanoma in a subject in need thereof, for example, PD-1 refractory melanoma, comprising administering to the subject a CD40 agonist such as sotigalimab, either as a standalone therapy or in combination with other therapeutic agents such as PD-1/PD-L1 inhibitor(s). Certain embodiments employ one or more biomarkers and/or other characteristics, for example, clinical histories, to further select patients that will benefit most from treatment with CD40 agonist therapy.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2, or 1% to a reference quantity, level, value, number, frequency, percentage, dimension, size, amount, weight, or length.

An “antagonist” or “inhibitor” refers to biological structure or chemical agent that interferes with or otherwise reduces the physiological action of another molecule, such as a protein. In some instances, the antagonist or inhibitor specifically binds to the other molecule and/or a functional ligand of the other molecule. In some instances, the antagonist or inhibitor down-regulates the expression of the other molecule. Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

An “antibody” or an “antigen binding fragment thereof” refers an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)2, Fv, single chain (scFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity.

The term “antigen-binding fragment” as used herein refers to a polypeptide fragment that contains at least one CDR of an immunoglobulin heavy and/or light chains that binds to the antigen of interest, in particular to CD40. In this regard, an antigen-binding fragment of the herein described antibodies may comprise 1, 2, 3, 4, 5, or all 6 CDRs of a VH and VL sequence.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an antibody is said to specifically bind an antigen when it preferentially recognizes its target antigen in a complex mixture of proteins and/or macromolecules.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv, single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments, etc., described herein under the definition of “antibody”.

The term “binding” refers to a direct association between two molecules, due to, for example, covalent, electrostatic, hydrophobic, and ionic and/or hydrogen-bond interactions, including interactions such as salt bridges and water bridges.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “half maximal effective concentration” or “EC50” refers to the concentration of an agent (e.g., CD40 agonist) as described herein at which it induces a response halfway between the baseline and maximum after some specified exposure time; the EC50 of a graded dose response curve therefore represents the concentration of a compound at which 50% of its maximal effect is observed. EC50 also represents the plasma concentration required for obtaining 50% of a maximum effect in vivo. Similarly, the “EC90” refers to the concentration of an agent or composition at which 90% of its maximal effect is observed. The “EC90” can be calculated from the “EC50” and the Hill slope, or it can be determined from the data directly, using routine knowledge in the art. In some embodiments, the EC50 of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC50 value of about 1 nM or less.

The “half maximal inhibitory concentration” (or “IC50”) is a measure of the potency of an agent in inhibiting a specific biological or biochemical function, for example, tumor growth. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC50 represents the concentration of an agent that is required for 50% inhibition in vitro. The IC50 of an agent can be determined by constructing a dose-response curve and examining the effect of different concentrations of the agent on the desired activity, for example, inhibition of tumor cell proliferation, tumor-cell killing.

The “half-life” of an agent refers to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing” or “enhancing” as well as “decreasing” or “reducing,” typically in a statistically significant or a physiologically significant amount or degree relative to a control. An “increased” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more relative to a control. An “increased” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount relative to a control. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount relative to a control. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount relative to a control. Examples of comparisons and “statistically significant” amounts are described herein.

“Pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example, which has been approved by the United States Food and Drug Administration as being acceptable for use in humans or domestic animals.

A “pharmaceutical composition” refers to a formulation of an agent (e.g., CD40 agonist) described herein and a medium generally accepted in the art for the delivery of the biologically active compound to mammals, including humans. Such a medium includes all pharmaceutically acceptable carriers, diluents, and excipients.

In certain embodiments, the “purity” of any given agent in a composition may be defined. For instance, certain compositions may comprise an agent that is at least 80%, 85%, 90%, 91%, 92%, 93%, 94%, 95%, 96%, 97%, 98%, 99%, or 100% pure on a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “polynucleotide” and “nucleic acid” includes mRNA, RNA, cRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given reference quantity, level, value, number, frequency, percentage, dimension, size, amount, weight, length, or other.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent (e.g., CD40 agonist) needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Methods and Compositions

sCD8 and CCL17. Certain embodiments relate to methods of treating melanoma in a human subject in need thereof, comprising administering to the subject a CD40 agonist, wherein the subject has increased soluble CD8 (sCD8) levels and decreased CCL17 levels, relative to a control or reference, thereby treating melanoma in the human subject in need thereof. In particular embodiments, the levels are protein levels.

CD8 (cluster of differentiation 8) refers to a transmembrane glycoprotein that serves as a co-receptor for the T-cell receptor (TCR) (see Uniprot: P10966). Soluble CD8 (sCD8) refers to a soluble or non-cell associated form of CD8 that can be found, for example, in blood or serum (see, for example, Zielinski et al., Clinical Immunology and Immunopathol. 57:74-82, 1990; and Ho et al., Blood. 84:3550-7, 1994). CCL17 refers to a chemokine produced in the thymus and by antigen-presenting cells like dendritic cells, macrophages, and monocytes, and is a chemotactic factor for T cells (see Uniprot: Q92583; and Weide et al., Cancer Immunol Immunother. 64:1075-82, 2015). In some embodiments, the subject has increased sCD8 serum protein levels and decreased CCL17 serum protein levels, relative to a control or reference.

Certain embodiments comprise (a) determining sCD8 and CCL17 levels in a tissue sample from the subject, and (b) administering the CD40 agonist to the subject if sCD8 levels in the tissue sample are increased and CCL17 levels in the tissue sample are decreased, relative to a control or reference

Levels of sCD8 and CCL17 can be determined according to routine techniques in the art. For example, certain embodiments comprise determining sCD8 and CCL17 protein levels in the tissue sample by an immunoassay, for example, by a high-throughput multiplex immunoassay (see Example 1), immunohistochemistry (IHC) such as chromogenic or fluorescent IHC, enzyme linked immunosorbent assay (ELISA), or Western blot. Specific embodiments comprise (a) determining sCD8 and CCL17 protein levels in a serum sample from the subject by a high-throughput multiplex immunoassay, and (b) administering the CD40 agonist to the subject if sCD8 protein levels in the serum sample are increased and CCL17 protein levels in the serum sample are decreased, relative to a control or reference.

CD4, CD40, and other Immune Markers. Some embodiments include methods of treating melanoma in a human subject in need thereof, comprising administering to the subject a CD40 agonist, wherein the subject has increased CD4, CD40, and CCL14 levels and decreased ABCF1, TP53, TCF3, and HDAC11 levels, relative to a control or reference, thereby treating melanoma in the human subject in need thereof.

CD4 refers to a glycoprotein that serves as a co-receptor for the T-cell receptor (TCR) (see Uniprot: P01730). CD40 refers to a costimulatory protein expressed, for example, on antigen-presenting cells (see Uniprot: P25942); the binding of CD154 (CD40L) on TH cells to CD40 activates antigen presenting cells and induces a variety of downstream effects. Chemokine (C—C motif) ligand 14 (CCL14) refers to a small cytokine belonging to the CC chemokine family (see Uniprot: Q16627).

ABCF1 (ATP-binding cassette sub-family F member 1) is a member of the superfamily of ATP-binding cassette (ABC) transporters, which transport various molecules across extra- and intra-cellular membranes (see Q8NE71). Tumor protein (TP53; also p53) refers to the full-length TP54 tumor suppressor protein and isoforms thereof (see Uniprot: P04637). Transcription Factor 3 (TCF3; E2A immunoglobulin enhancer-binding factors E12/E47) refers to a member of the E protein (class I) family of helix-loop-helix transcription factors (see Uniprot: P15923). Histone deacetylase 11 (HDAC11) refers to a histone deacetylase enzyme that in humans is encoded by the HDAC11 gene on chromosome 3 (see Uniprot: Q96DB2), which control DNA expression by modifying the core histone octamers that package DNA into dense chromatin structures and repress gene expression.

In some embodiments, the subject has increased CD4, CD40, and CCL14 RNA and/or protein levels and decreased ABCF1, TP53, TCF3, and HDAC11 RNA and/or protein levels in melanoma tissue, relative to a control or reference.

Some embodiments include (a) determining CD4, CD40, CCL14, ABCF1, TP53, TCF3, and HDAC11 levels in a tissue sample from the subject, and (b) administering the CD40 agonist to the subject if CD4, CD40, and CCL14 levels in the tissue sample are increased and ABCF1, TP53, TCF3, and HDAC11 levels in the tissue sample are decreased, relative to a control or reference.

The expression levels of CD4, CD40, CCL14, ABCF1, TP53, TCF3, and HDAC11 in a tissue sample can be determined by a variety of techniques in the art. Certain methods include determining RNA expression levels, for example, by a nucleic acid hybridization assay (such as sandwich hybridization assay, competitive hybridization assay, nuclease hybridization assay, hybridization-ligation assay, or dual ligation hybridization assay) or RNA sequencing (RNA-Seq). Some methods include determining protein expression levels, for example, by an immunoassay such as a high-throughput multiplex immunoassay, immunohistochemistry (IHC) such chromogenic or fluorescent IHC, enzyme linked immunosorbent assay (ELISA), or Western blot.

Specific embodiments include determining CD4, CD40, CCL14, ABCF1, TP53, TCF3, and HDAC11 RNA expression levels in a melanoma biopsy sample from the subject, optionally a FFPE biopsy sample, and (b) administering the CD40 agonist to the subject if CD4, CD40, and CCL14 levels in the tissue sample are increased and ABCF1, TP53, TCF3, and HDAC11 levels in the tissue sample are decreased, relative to a control or reference.

PD-L1 Negative Melanoma. Certain embodiments include method of treating PD-L1 negative melanoma in a human subject in need thereof, comprising administering to the subject a CD40 agonist, optionally in combination with a PD-1/PD-L1 inhibitor, thereby treating the PD-L1 negative melanoma in the human subject in need thereof.

Programmed cell death protein 1 (PD-1; CD279 (cluster of differentiation 279)) refers a protein expressed on the surface of cells that regulates the immune response to the cells of the human body, for example, by down-regulating the immune system and promoting self-tolerance by suppressing T cell inflammatory activity (see Uniprot: Q15116). PD-1 is an immune checkpoint that promotes apoptosis of antigen-specific T-cells in lymph nodes, and reduces apoptosis in regulatory T cells (anti-inflammatory, suppressive T cells). Programmed death-ligand 1 (PD-L1) is a 40 kDa type 1 transmembrane protein that binds to its receptor, PD-1, which is expressed on activated T cells, B cells, and myeloid cells, to modulate activation or inhibition (see Uniprot: Q9NZQ7). The term “PD-L1 negative melanoma” refers to a melanoma in which PD-L1 levels are absent (i.e., undetectable or substantially undetectable), or significantly decreased relative to a control or reference.

Certain embodiments thus comprise administering the CD40 agonist and optional PD-1/PD-L1 inhibitor to the subject if PD-L1 levels in the tissue sample are absent (i.e., undetectable or substantially undetectable) or decreased relative to that of a control or reference. Certain embodiments comprise administering the CD40 agonist and optional PD-1/PD-L1 inhibitor to the subject if the PD-L1 expression levels in the in the tissue sample are decreased by a statistically significant amount relative to the PD-L1 levels of the control or reference. Specific embodiments comprise administering the CD40 agonist and optional PD-1/PD-L1 inhibitor to the subject if PD-L1 expression levels in the in the tissue sample are decreased by about or at least about 2, 3, 4, 5, 6, 7, 8, 9, or 10, 50, or 100-fold or more relative to the PD-L1 levels of the control or reference.

PD-L1 expression levels in a sample of tissue (e.g., cancer tissue) can be determined by any variety of methods. For example, PD-L1 protein levels can be determined by immunohistochemistry (IHC) including chromogenic or fluorescent IHC, enzyme linked immunosorbent assay (ELISA), or Western blot on a human AR protein or gene, among other assays. PD-L1 mRNA levels can be measured, for example, by RT-PCR, for example, quantitative competitive (QC) RT-PCR, among other techniques known in the art. Certain embodiments thus include the step of determining or detecting or measuring PD-L1 levels in a tissue sample from a subject in need thereof. Also included is the step of comparing the PD-L1 levels in a tissue sample relative to that of a control or reference.

Certain embodiments thus include (a) determining PD-L1 levels in a tissue sample from the subject, and (b) administering the CD40 agonist and optional PD-1/PD-L1 inhibitor to the subject if PD-L1 levels in the tissue sample are absent (undetectable) or decreased relative to a control or reference. In specific embodiments, (a) comprises determining PD-L1 levels in the tissue sample by an immunoassay, including immunohistochemistry (IHC) such as chromogenic or fluorescent IHC, enzyme linked immunosorbent assay (ELISA), or Western blot, or by flow cytometry.

Specific embodiments include (a) determining PD-L1 levels by IHC in a melanoma biopsy sample from the subject, and (b) administering the CD40 agonist and optional PD-1/PD-L1 inhibitor to the subject if PD-L1 levels in the melanoma biopsy sample are absent (undetectable) or decreased relative to a control or reference.

Exemplary PD-1/PD-L1 inhibitors for use in combination with CD40 agonist therapy are known in the art. For instance, in some embodiments, the PD-1 inhibitor is selected from nivolumab, pembrolizumab, cemiplimab, JTX-4014, spartalizumab, camrelizumab, sintilimab, tislelizumab, toripalimab, dostarlimab, MGA012, AMP-22, and AMP-514. In some embodiments, the PD-L1 inhibitor is selected from atezolizumab, avelumab, and durvalumab

Prior anti-CTLA4 Therapy. Certain embodiments include methods of treating melanoma in a human subject in need thereof, comprising administering to the subject a CD40 agonist, wherein the subject has previously undergone, and is no longer undergoing, treatment with a CTLA-4 inhibitor, thereby treating the melanoma in the human subject in need thereof.

Cytotoxic T-lymphocyte-associated protein 4 (CTLA-4, or CD152 (cluster of differentiation 152), is a protein receptor that functions as an immune checkpoint and downregulates immune responses (see Uniprot: P16410). CTLA-4 inhibitor therapy is used to inhibit immune system tolerance to tumors and thereby provide a potentially useful immunotherapy strategy for patients with cancer (see, for example, Pardoll, Nat Rev Cancer. 12:252-64, 2012; and Syn et al., Lancet Oncol. 18: e731-e741, 2017). Examples of CTLA inhibitors include ipilimumab and tremelimumab.

Certain embodiments define an optimal time period between the end of the prior CTLA-4 inhibitor therapy and the beginning of the present CD40 agonist therapy. For instance, some embodiments comprising administering the CD40 agonist (for example, as in initial or first dosage) at least about 1, 2, 3, 4, 5, 10, 20, 30, 40, 50, 60, 70, 80, 90, 80, 90, 100, 150, 200, 250, 300, 350, 400, 450, 500, 550, or 600 days or more after the end of treatment with the CTLA-4 inhibitor, for example, between about 100-200 days, 100-300 days, 100-400 days, 100-500 days, or 100-600 days after the end of treatment with the CTLA-4 inhibitor, or between about 200-300 days, 200-400 days, 200-500 days, or 200-600 days after the end of treatment with the CTLA-4 inhibitor, or between about 300-400 days, 300-500 days, or 300-600 days after the end of treatment with the CTLA-4 inhibitor, or between about 400-500 days or 400-600 days after the end of treatment with the CTLA-4 inhibitor, or between about 500-600 days after the end of treatment with the CTLA-4 inhibitor.

In certain embodiments, the prior CTLA-4 inhibitor therapy increases the response rate to CD40 agonist therapy. The term response rate, or objective response rate (ORR), refers to ORR is defined as the proportion of subjects with tumor size reduction of a predefined amount and for a minimum time period (see, for example, Eisenhauer et al., Eur J Cancer. 45:228-247, 2009). In some instances, the ORR is defined as the sum of partial responses plus complete response (CR), the latter being defined as no detectable evidence of tumor. Thus, in some embodiments, prior treatment with the CTLA-4 inhibitor increases the response rate to the CD40 agonist, relative to no prior treatment with a CTLA-4 inhibitor.

Generally, the methods and compositions described herein can employ any variety of CD40 agonists (see, for example, Djureinovic et al., Cancers (Basel). 13 (6): 1302, 2021). General examples of CD40 agonists include antibodies, and antigen binding fragments thereof, which specifically bind to and agonize human CD40, and small molecule CD40 agonists. In particular embodiments, the CD40 agonist is selected from sotigalimab (APX005M), ChiLob7/4, ADC-1013, SEA-CD40, selicrelumab, and CDX-1140. In specific embodiments, the CD40 agonist is sotigalimab (APX005M; see also U.S. Pat. No. 9,676,861; and O'Hara et al., Lancet Oncol. 22 (1): 118-131, 2021).

In certain embodiments, the methods and compositions described herein are for treating a subject with melanoma, including PD-1 refractory melanoma. The term “PD-1 refractory melanoma” includes a melanoma that does not significantly respond to, has previously failed to respond to, or has become non-responsive (e.g., via selection) to, therapy with a PD-1/PD-L1 inhibitor, for example, prior therapy with a PD-1/PD-L1 inhibitor. Certain embodiments define an optimal time period between the end of the prior PD-1/PD-L1 inhibitor therapy and the beginning of the present CD40 agonist therapy (or the present combination therapy, for example, a combination comprising a CD40 agonist and a PD-1/PD-L1 inhibitor). For instance, some embodiments comprising administering the CD40 agonist or combination (for example, as in initial or first dosage) at least about 1, 2, 3, 4, 5, 10, 20, 30, 40, 50, 60, 70, 80, 90, 80, 90, 100, 150, 200, 250, or 300 days or more after the end of treatment with the prior PD-1/PD-L1 inhibitor. Examples of PD-1 inhibitors include nivolumab, pembrolizumab, cemiplimab, JTX-4014, spartalizumab, camrelizumab, sintilimab, tislelizumab, toripalimab, dostarlimab, MGA012, AMP-22, and AMP-514. Examples of PD-L1 inhibitors include atezolizumab, avelumab, and durvalumab. In specific embodiments, the prior PD-1 inhibitor is nivolumab and the present CD40 agonist is sotigalimab, optionally in combination with nivolumab. In specific embodiments, the prior PD-1 inhibitor is pembrolizumab and the present CD40 agonist is sotigalimab, optionally in combination with nivolumab.

Examples of a “reference” include a value, amount, sequence, or other characteristic obtained, for example, from a database. A “reference” also includes value, amount, sequence, or other characteristic obtained from a non-cancerous tissue from one or more controls, for example, one or more healthy or non-cancerous control subjects (e.g., a population of healthy or non-cancerous control subjects), or one or more corresponding non-cancerous control tissues from the subject being tested.

In some embodiments, the tissue sample is a liquid biopsy (for example, a blood sample, a serum sample), a surgical sample, or other biopsy sample obtained from the subject, including a biopsy of melanoma tissue. In specific embodiments, the tissue sample is a formalin fixed paraffin embedded (FFPE) tissue sample. Certain embodiments include the step of obtaining the tissue sample from the subject, for example, prior to determining biomarker levels or status.

In some embodiments, the subject is a human subject.

In certain embodiments, the methods and compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein increase cancer cell-killing in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the methods and compositions described herein increase progression-free survival, overall survival, and/or survival post-progression in the subject in need thereof, for example, by about or at least about 2, 3, 4, 5, 6, 7, 8, 9, 10, 11, 12, 13, 14, 15, 16, 17, 18, 19, 20, 21, 22, 23, or 24 months or more, or by about or at least about 1, 2, 3, 4, 5, 6, 7, 8, 9, or 10 years or more. In certain embodiments, the methods and compositions described are sufficient to result in stable disease. In certain embodiments, the methods and compositions described herein are sufficient to result in clinically relevant reduction in symptoms of a particular disease indication known to the skilled clinician.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents (e.g., CD40 agonists) described herein are generally incorporated into one or more therapeutic or pharmaceutical compositions prior to administration. A pharmaceutical composition comprises one or more of the agents described herein in combination with a physiologically acceptable carrier or excipient as described elsewhere herein.

To prepare a pharmaceutical composition, an effective amount of one or more of the agents is mixed with any pharmaceutical carrier(s) or excipient known to those skilled in the art to be suitable for the particular mode of administration. A pharmaceutical carrier may be liquid, semi-liquid or solid. Solutions or suspensions may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered parenterally (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

The compositions comprising agents (e.g., CD40 agonists) may be prepared with carriers that protect the agent against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Administration of an agent described herein, in pure form or in an appropriate therapeutic or pharmaceutical composition, can be carried out via any of the accepted modes of administration of agents for serving similar utilities. Examples include administering the pharmaceutical composition to the subject by systemic administration, intratumoral injection, or intranasal administration, for example, parenteral administration, including intravenous and subcutaneous administration. Preferred modes of administration depend upon the nature of the condition to be treated or prevented. Particular embodiments include administration by IV infusion.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1: CD40 Agonist Melanoma Cohort Biomarker Summary

Human clinical trials were conducted to study the efficacy of the CD40 agonist sotigalimab (APX005M) in melanoma patients, and biomarkers and clinical histories in the cohort were analyzed to identify highest responders to sotigalimab therapy.

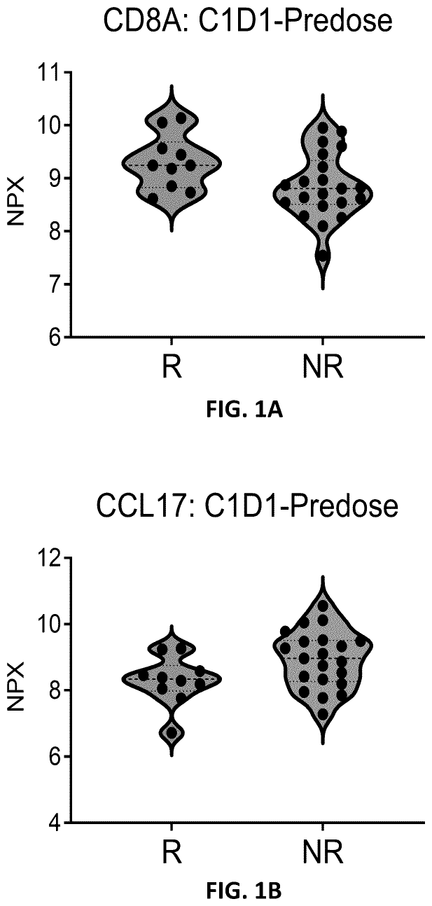

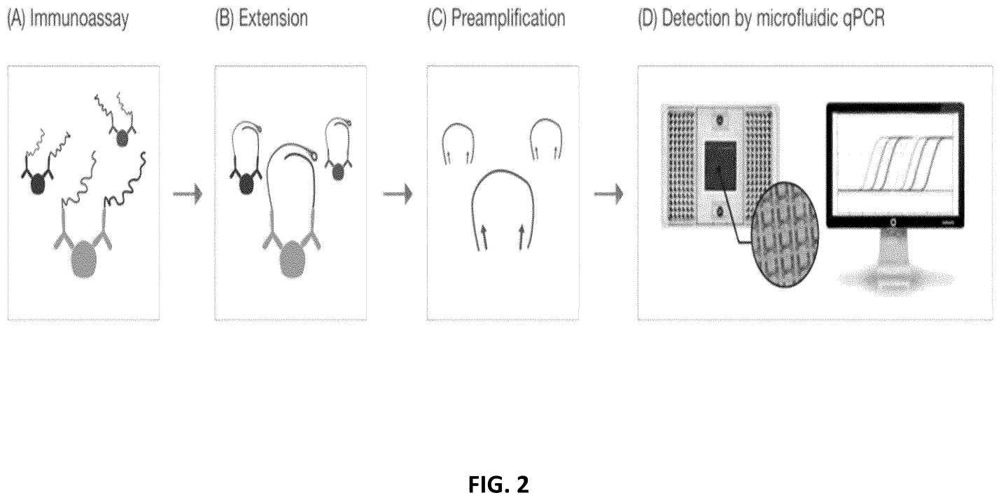

As shown in FIGS. 1A-1B, increased sCD8A and decreased CCL17 correlate with tumor responsiveness to sotigalimab therapy (R, responder; NR, non-responder). Here, serum proteins were analyzed by Olink Technology, a high-throughput, multiplex immunoassays that measure numerous proteins simultaneously using only one microliter of serum. FIG. 2 shows the immunoassay format. (A) 92 Probes (antibody pairs, labeled with DNA oligos) bind target antigen in solution; (B) Oligonucleotides are brought into proximity to hybridize, and are extended by a DNA polymerase (specificity); (C) the newly created pieces of DNA barcode are amplified by PCR (sensitivity); (D) the amount of each DNA barcode is quantified by microfluidic qPCR, and the protein levels are expressed in NPX units, Olink's arbitrary unit in Log 2 scale.

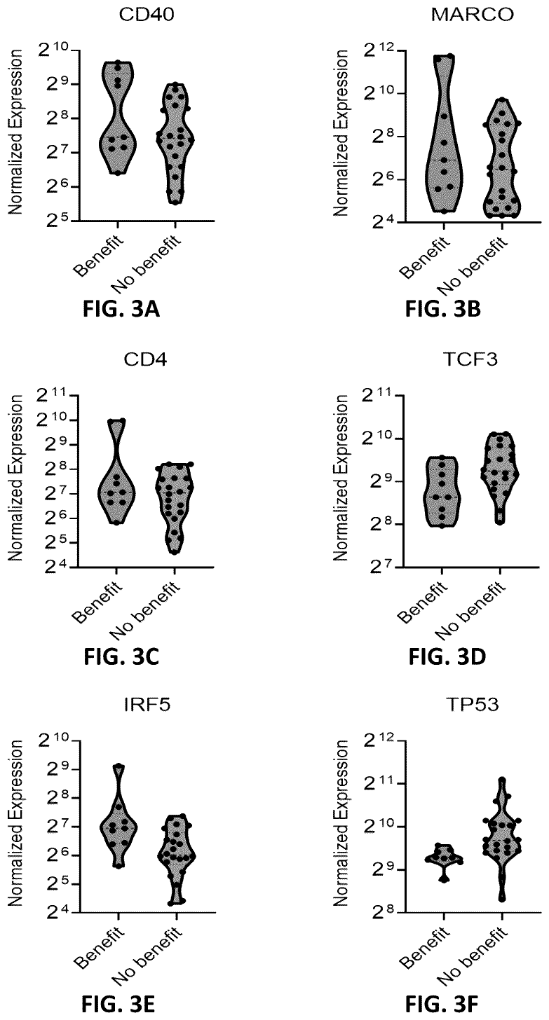

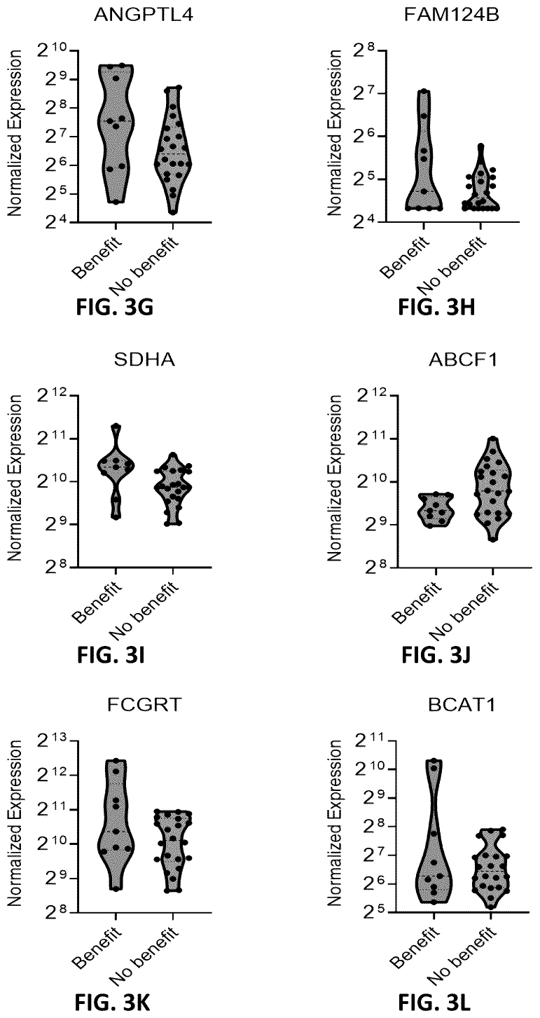

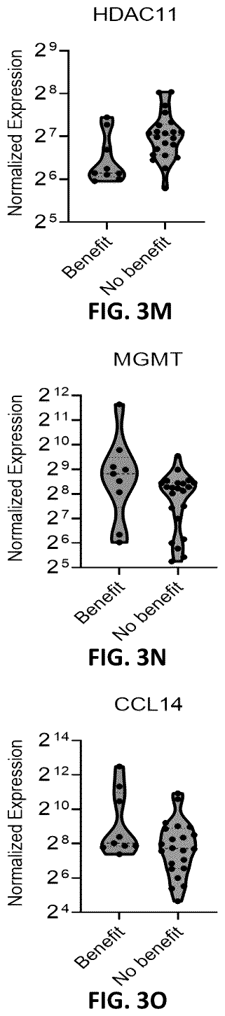

As shown in Table E1 and FIGS. 3A-30, increased expression of CD4, CD40, and CCL14 and decreased expression of ABCF1, TP53, TCF3, and HDAC11 correlate with tumor responsiveness to sotigalimab therapy. Here, FFPE biopsies were analyzed for gene expression by NanoString panels (IO pan cancer 360 panel/770 genes). Results are based p-values by TTEST.

TABLE E1

| | | |
|---|---|---|
| Gene | P value | Fold change |

“UP” in Benefitting Patients

| | | |
|---|---|---|
| CCL14 | 0.049 | 2.6 |
| MARCO | 0.038 | 2.1 |
| IRF5 | 0.018 | 1.9 |
| ANGPTL4 | 0.020 | 1.9 |
| MGMT | 0.045 | 1.9 |
| CD4 | 0.042 | 1.7 |
| CD40 | 0.048 | 1.6 |
| BCAT1 | 0.038 | 1.5 |
| FCGRT | 0.029 | 1.5 |
| FAM124B | 0.033 | 1.4 |
| SDHA | 0.024 | 1.3 |

“DOWN” in Benefitting Patients

| | | |
|---|---|---|
| ABCF1 | 0.036 | 0.7 |
| TP53 | 0.036 | 0.7 |
| TCF3 | 0.028 | 0.7 |
| HDAC11 | 0.044 | 0.7 |

As shown in Table E2, PD-L1 negative status correlates with tumor responsiveness to sotigalimab in combination with nivolumab. PD-L1 immunohistochemistry (IHC) status was measured using the 28-8 clone for Tumor Proportion Score; two results from prior PD-L1 testing were used when baseline results for the current study were unavailable.

TABLE E2
PD-L1 Status

| | Positive (≥1%) | Negative (<1%) | | Unable to score | | Not tested | |
|---|---|---|---|---|---|---|---|
| PR | 2 (25%) | 3 | (14.3%) | 1 | (50%) | 0 | (0%) |
| SD | 2 (25%) | 5 | (23.8%) | 1 | (50%) | 1 | (14.3%) |
| PD | 4 (50%) | 11 | (52.4%) | 0 | (0%) | 4 | (57.1%) |
| NE | 0 (0%) | 2 | (9.5%) | 0 | (0%) | 2 | (28.6%) |

| | | | | |
|---|---|---|---|---|
| Total | 8 | 21 | 2 | 7 |

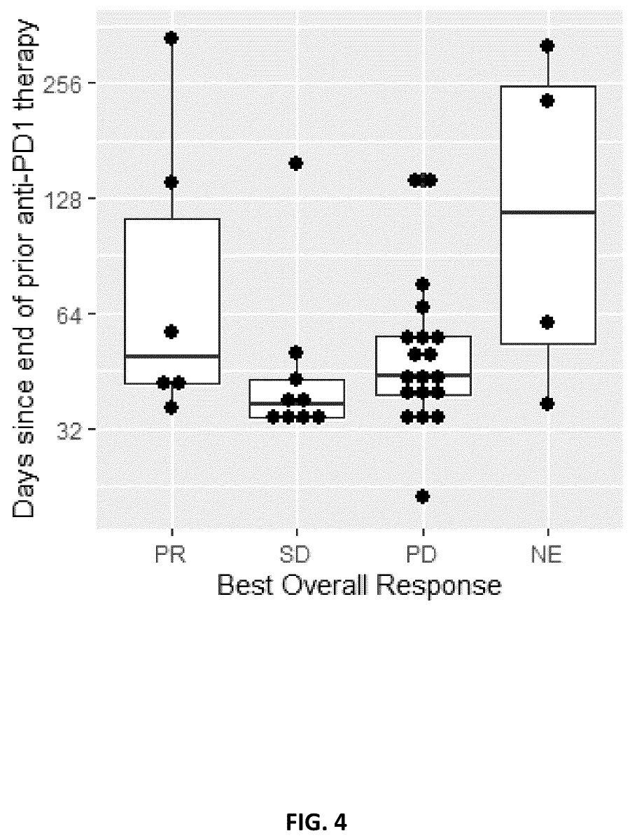

Table E3 and FIG. 4 show the tumor responsiveness in subjects having undergone prior therapy with nivolumab or pembrolizumab. Here, subjects with PD-1 refractory melanoma were treated with sotigalimab in combination with nivolumab.

TABLE E3

| | |
|---|---|
| Prior Nivolumab | Prior Pembrolizumab |

| | | | | |
|---|---|---|---|---|
| PR | 4 | (13.8%) | 2 | (22.2%) |
| SD | 6 | (20.7%) | 3 | (33.3%) |
| PD | 15 | (51.7%) | 4 | (44.4%) |
| NE | 4 | (13.8%) | 0 | (0%) |

| | | |
|---|---|---|
| Total | 29 | 9 |

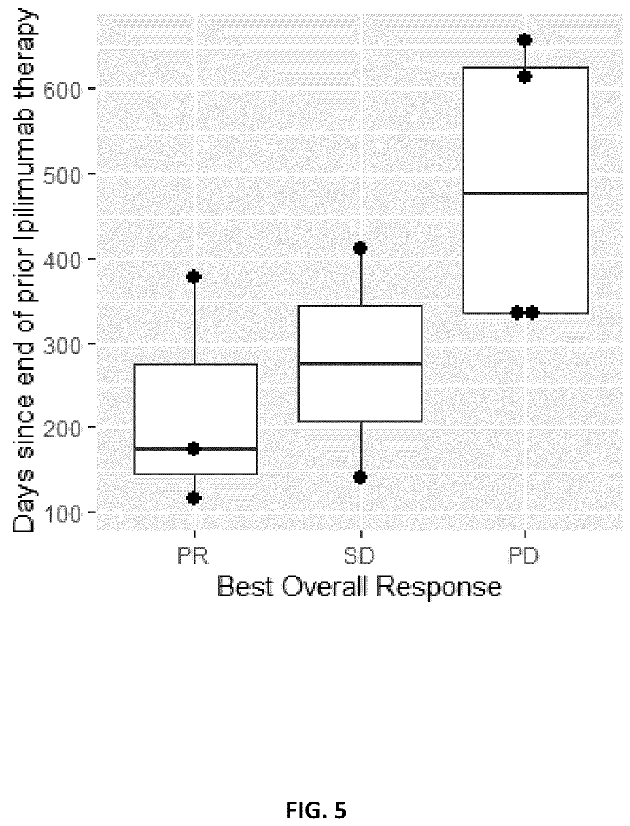

As shown in Table E4 and FIG. 5, prior anti-CTLA4 (Ipilimumab) therapy/proximity correlates with tumor responsiveness to sotigalimab therapy.

TABLE E4

| | |
|---|---|
| Prior Ipilimumab | No prior Ipilimumab |

| | | | | |
|---|---|---|---|---|
| PR | 3 | (33.3%) | 3 | (10.3%) |
| SD | 2 | (22.2%) | 7 | (24.1%) |
| PD | 4 | (44.4%) | 15 | (51.7%) |
| NE | 0 | (0%) | 4 | (13.8%) |

| | | |
|---|---|---|
| Total | 9 | 29 |

Abbreviations: PR (partial response), SD (stable disease), PD (Progressive disease), NE (no evidence of disease).

1. A method of treating melanoma in a human subject in need thereof, comprising
administering to the subject a CD40 agonist, wherein the subject has increased soluble CD8 (sCD8) levels and decreased CCL17 levels, relative to a control or reference,
thereby treating melanoma in the human subject in need thereof.

2. The method of claim 1, wherein the subject has increased sCD8 serum protein levels and decreased CCL17 serum protein levels, relative to a control or reference.

3. The method of claim 1 or 2, comprising:
(a) determining sCD8 and CCL17 levels in a tissue sample from the subject, and
(b) administering the CD40 agonist to the subject if sCD8 levels in the tissue sample are increased and CCL17 levels in the tissue sample are decreased, relative to a control or reference.

4. The method of claim 3, wherein (a) comprises determining sCD8 and CCL17 protein levels in the tissue sample by an immunoassay, optionally by a high-throughput multiplex immunoassay, immunohistochemistry (IHC) such as chromogenic or fluorescent IHC, enzyme linked immunosorbent assay (ELISA), or Western blot.

5. The method of claim 3 or 4, wherein the tissue sample is a liquid biopsy optionally a blood or serum sample, a surgical sample, or other biopsy sample obtained from the subject, optionally a biopsy of melanoma tissue.

6. The method of any one of claims 3-5, comprising obtaining or receiving the tissue sample from the subject in need thereof.

7. The method of any one of claims 3-6, comprising
(a) determining sCD8 and CCL17 protein levels in a serum sample from the subject by a high-throughput multiplex immunoassay, and
(b) administering the CD40 agonist to the subject if sCD8 protein levels in the serum sample are increased and CCL17 protein levels in the serum sample are decreased, relative to a control or reference.

8. The method of any one of claims 1-7, wherein the CD40 agonist is selected from sotigalimab (APX005M), ChiLob7/4, ADC-1013, SEA-CD40, selicrelumab, and CDX-1140.

9. The method of any one of claims 1-8, wherein the melanoma is PD-1 refractory melanoma.

10. A method of treating melanoma in a human subject in need thereof, comprising
administering to the subject a CD40 agonist, wherein the subject has increased CD4, CD40, and CCL14 levels and decreased ABCF1, TP53, TCF3, and HDAC11 levels, relative to a control or reference,
thereby treating melanoma in the human subject in need thereof.

11. The method of claim 10, wherein the subject has increased CD4, CD40, and CCL14 RNA levels and decreased ABCF1, TP53, TCF3, and HDAC11 RNA levels in melanoma tissue, relative to a control or reference.

12. The method of claim 10 or 11, comprising
(a) determining CD4, CD40, CCL14, ABCF1, TP53, TCF3, and HDAC11 levels in a tissue sample from the subject, and
(b) administering the CD40 agonist to the subject if CD4, CD40, and CCL14 levels in the tissue sample are increased and ABCF1, TP53, TCF3, and HDAC11 levels in the tissue sample are decreased, relative to a control or reference.

13. The method of claim 12, wherein (a) comprises determining CD4, CD40, CCL14, ABCF1, TP53, TCF3, and HDAC11 RNA expression levels, optionally by a nucleic acid hybridization assay (such as sandwich hybridization assay, competitive hybridization assay, nuclease hybridization assay, hybridization-ligation assay, or dual ligation hybridization assay) or RNA sequencing (RNA-Seq).

14. The method of claim 12, wherein (a) comprises determining CD4, CD40, CCL14, ABCF1, TP53, TCF3, and HDAC11 protein levels, optionally by an immunoassay, optionally by a high-throughput multiplex immunoassay, immunohistochemistry (IHC) such chromogenic or fluorescent IHC, enzyme linked immunosorbent assay (ELISA), or Western blot.

15. The method of any one of claims 12-14, wherein the tissue sample is a liquid biopsy optionally a blood or serum sample, a surgical sample, or other biopsy sample obtained from the subject, optionally a biopsy of melanoma tissue, including a formalin fixed paraffin embedded (FFPE) tissue sample.

16. The method of any one of claims 12-15, comprising obtaining or receiving the tissue sample from the subject in need thereof.

17. The method of any one of claims 12-16, comprising
(a) determining CD4, CD40, CCL14, ABCF1, TP53, TCF3, and HDAC11 RNA expression levels in a melanoma biopsy sample from the subject, optionally a FFPE biopsy sample, and
(b) administering the CD40 agonist to the subject if CD4, CD40, and CCL14 levels in the tissue sample are increased and ABCF1, TP53, TCF3, and HDAC11 levels in the tissue sample are decreased, relative to a control or reference.

18. The method of any one of claims 10-17, wherein the CD40 agonist is selected from sotigalimab (APX005M), ChiLob7/4, ADC-1013, SEA-CD40, selicrelumab, and CDX-1140.

19. The method of any one of claims 10-18, wherein the melanoma is PD-1 refractory melanoma.

20. A method of treating PD-L1 negative melanoma in a human subject in need thereof, comprising
administering to the subject a CD40 agonist, optionally in combination with and a PD-1/PD-L1 inhibitor,
thereby treating the PD-L1 negative melanoma in the human subject in need thereof.

21. The method of claim 20, comprising
(a) determining PD-L1 levels in a tissue sample from the subject, and
(b) administering the CD40 agonist or optional combination to the subject if PD-L1 levels in the tissue sample are absent (undetectable) or decreased relative to a control or reference.

22. The method of claim 21, wherein (a) comprises determining PD-L1 levels in the tissue sample by an immunoassay, optionally immunohistochemistry (IHC) such as chromogenic or fluorescent IHC, enzyme linked immunosorbent assay (ELISA), or Western blot, or by flow cytometry.

23. The method of any one of claims 21-22, comprising
(a) determining PD-L1 levels by IHC in a melanoma biopsy sample from the subject, and
(b) administering the CD40 agonist or optional combination to the subject if PD-L1 levels in the melanoma biopsy sample are absent (undetectable) or decreased relative to a control or reference.

24. The method of any one of claims 21-23, wherein the tissue sample is a liquid biopsy optionally a blood or serum sample, a surgical sample, or other biopsy sample obtained from the subject, optionally a biopsy of melanoma tissue, including a formalin fixed paraffin embedded (FFPE) tissue sample.

25. The method of any one of claims 21-24, comprising obtaining or receiving the tissue sample from the subject in need thereof.

26. The method of any one of claims 20-25, wherein the CD40 agonist is selected from sotigalimab (APX005M), ChiLob7/4, ADC-1013, SEA-CD40, selicrelumab, and CDX-1140.

27. The method of any one of claims 20-26, wherein the PD-1 inhibitor is selected from nivolumab, pembrolizumab, cemiplimab, JTX-4014, spartalizumab, camrelizumab, sintilimab, tislelizumab, toripalimab, dostarlimab, MGA012, AMP-22, and AMP-514.

28. The method of any one of claims 20-26, wherein the PD-L1 inhibitor is selected from atezolizumab, avelumab, and durvalumab.

29. The method of any one of claims 20-28, wherein the melanoma is PD-1 refractory melanoma.

30. A method of treating melanoma in a human subject in need thereof, comprising
administering to the subject a CD40 agonist, wherein the subject has previously undergone, and is no longer undergoing, treatment with a CTLA-4 inhibitor,
thereby treating the melanoma in the human subject in need thereof.

31. The method of claim 30, comprising administering the CD40 agonist at least about 1, 2, 3, 4, 5, 10, 20, 30, 40, 50, 60, 70, 80, 90, 80, 90, 100 days or more after the end of treatment with the CTLA-4 inhibitor, optionally between about 100-200 days, 100-300 days, 100-400 days, 100-500 days, or 100-600 days after the end of treatment with the CTLA-4 inhibitor.

32. The method of claim 30, comprising administering the CD40 agonist at least about 200 days or more after the end of treatment with the CTLA-4 inhibitor, optionally between about 200-300 days, 200-400 days, 200-500 days, or 200-600 days after the end of treatment with the CTLA-4 inhibitor.

33. The method of claim 30, comprising administering the CD40 agonist at least about 300 days or more after the end of treatment with the CTLA-4 inhibitor, optionally between about 300-400 days, 300-500 days, or 300-600 days after the end of treatment with the CTLA-4 inhibitor.

34. The method of claim 30, comprising administering the CD40 agonist at least about 400 days or more after the end of treatment with the CTLA-4 inhibitor, optionally between about 400-500 days or 400-600 days after the end of treatment with the CTLA-4 inhibitor.

35. The method of claim 30, comprising administering the CD40 agonist at least about 500 days or more after the end of treatment with the CTLA-4 inhibitor, optionally between about 500-600 days after the end of treatment with the CTLA-4 inhibitor.

36. The method of claim 30, comprising administering the CD40 agonist at least about 600 days or more after the end of treatment with the CTLA-4 inhibitor.

37. The method of any one of claims 30-36, wherein prior treatment with the CTLA-4 inhibitor increases the response rate to the CD40 agonist, relative to no prior treatment with a CTLA-4 inhibitor.

38. The method of any one of claims 30-37, wherein the CD40 agonist is selected from sotigalimab (APX005M), ChiLob7/4, ADC-1013, SEA-CD40, selicrelumab, and CDX-1140.

39. The method of any one of claims 30-38, wherein the CTLA inhibitor is selected from ipilimumab and tremelimumab.

40. The method of any one of claims 30-39, wherein the melanoma is PD-1 refractory melanoma.
